(19) United States
(12) Patent Application Publication
(10) Pub. No.: US 2011/0257758 A1
(43) Pub. Date: Oct. 20, 2011

(54) SPLINED TAPERED MODULAR DISTAL STEM WITH PROXIMAL POROUS COATING

(75) Inventors: Aaron P. Smith, Warsaw, IN (US); Eric J. Fontenot, Frisco, IN (US); Keith R. Berend, New Albery, OH (US); Hugh Apthorp, East Sussex (GB); Ian Stockley, Sheffield (GB)

(73) Assignee: Biomet Manufacturing Corp., Warsaw, IN (US)

(21) Appl. No.: 13/038,782

(22) Filed: Mar. 2, 2011

Related U.S. Application Data
(60) Provisional application No. 61/310,865, filed on Mar. 5, 2010.

(51) Int. Cl. A61F 2/32

(52) U.S. Cl. 623/22.42

(57) ABSTRACT

A modular femoral hip joint prosthesis can include a proximal femoral body having a distal end and a proximal end. The proximal end can be adapted to mate with a femoral head. A femoral stem can have a proximal section and a distal section. The femoral stem can be adapted to matingly connect with the distal end of the proximal femoral body. The proximal section can have a porous metal coating that is adapted to facilitate bony ingrowth. The distal section can have at least one spline formed along an outer surface.

### CROSS-REFERENCE TO RELATED APPLICATIONS

This application claims the benefit and priority of 61/310,865, filed Mar. 5, 2010. The entire disclosure of the above application is incorporated herein by reference.

### FIELD

The present disclosure relates generally to femoral hip stems and more specifically to a modular femoral hip stem assembly that incorporates a distal stem having a proximal section that is porous coated and a distal tapered section that incorporates splines thereon.

### BACKGROUND

This section provides background information related to the present disclosure which is not necessarily prior art.

In some examples of femoral revision surgery, it may be required to repair a fractured or osteotomized femur around a seated femoral implant, such as by using cables or other known mechanical fixation. In one example, a surgeon can distally pot a femoral hip stem into an intact distal femur and subsequently wrap the non-intact proximal femur around the proximal portion of the femoral hip stem.

### SUMMARY

This section provides a general summary of the disclosure, and is not a comprehensive disclosure of its full scope or all of its features.

A modular femoral hip joint prosthesis can include a proximal femoral body having a distal end and a proximal end. The proximal end can be adapted to mate with a femoral head. A femoral stem can have a proximal section and a distal section. The femoral stem can be adapted to matingly connect with the distal end of the proximal femoral body. The proximal section can have a porous metal coating that is adapted to facilitate bony ingrowth. The distal section can have at least one spline formed along an outer surface.

According to additional features, the proximal section of the femoral stem can extend along a first axis and the distal section of the femoral stem can extend along a second axis. The first and the second axes can be non-parallel. The distal section of the femoral stem can taper toward a distal tip. The first and second axes can extend at an angle substantially between 2° and 10°. In one example, the angle can be 5°. The proximal section of the femoral stem can include a male taper end. The distal end of the proximal femoral body can include a female taper. The male taper end of the femoral stem can be adapted to be received in the female taper of the proximal femoral body. According to additional features, the modular femoral hip prosthesis can further comprise a fastener that is operable to extend into an opening in the proximal femoral and threadably mate with a threaded bore formed on the male taper end of the femoral stem.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of an exemplary modular femoral hip joint prosthesis constructed in accordance to one example of the present teachings and shown in an assembled position;

FIG. 2 is an exploded perspective view of the modular femoral hip joint prosthesis shown in FIG. 1; and

FIG. 3 is a medial view of the modular femoral hip joint prosthesis shown cooperating with a cerclage fixation assembly in an implanted position according to one example of the present teachings.

Corresponding reference numerals indicate corresponding parts throughout the several views of the drawings.

### DETAILED DESCRIPTION

Example embodiments will now be described more fully with reference to the accompanying drawings.

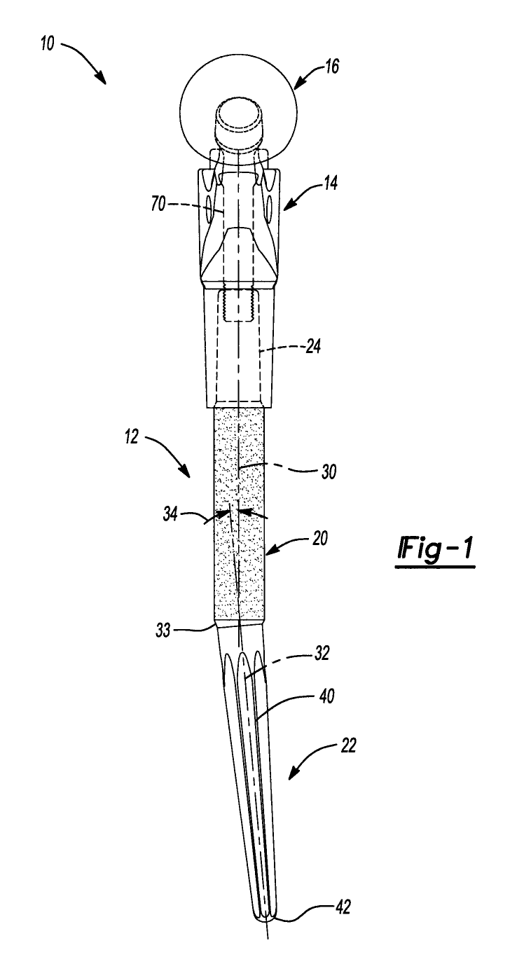

With initial reference to FIG. 1, a modular femoral hip joint prosthesis constructed in accordance to one example of the present teachings is shown and generally identified at reference numeral 10. The modular femoral hip joint prosthesis can generally comprise a distal stem 12, a proximal femoral body 14, and a femoral head 16. As will be described herein, the distal stem 12, proximal femoral body 14, and the femoral head 16 can all be modular components that may be selectively attached to each other during a surgical procedure, such as a femoral revision procedure. In this regard, each of the distal stem 12, the proximal femoral body 14 and the femoral head 16 can comprise a plurality of unique components having various dimensions that may be suitable for a particular patient. As such, a surgeon may have a kit of components including various distal stems, proximal femoral bodies and femoral heads all having various dimensions and features that the surgeon can intraoperatively select according to a patient's particular need.

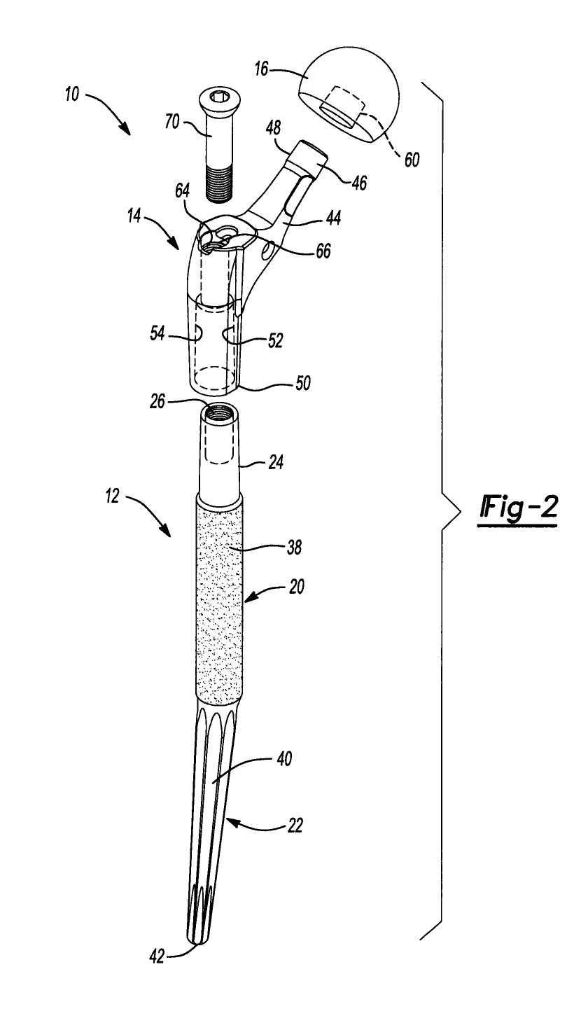

With continued reference to FIG. 1 and additional reference now to FIG. 2, the modular femoral hip joint prosthesis 10 will be further described. The distal stem 12 can include a proximal section 20 and a distal section 22. A male taper end 24 can extend from the proximal section 20. The male taper end 24 can generally define a blind threaded bore 26 formed therein. The proximal section 20 can generally be formed along a first axis 30. The distal section 22 can be generally formed along a second axis 32. In one example, a small angled transition section 33 (FIG. 1) may be formed between the proximal section 20 and the distal section 22. The respective first and second axes 30 and 32 can form an angle 34 relative to each other. The angle 34 can be any suitable angle such as between 2° and 10° for example and more specifically about 5°.

It is contemplated that a series of stems 12 can be provided that may each define a distinct angle 34, such that a surgeon can select the appropriate stem 12 having an angle that is best suited for a particular patient's needs. Moreover, each distal stem 12 can have distinct lengths. It is also contemplated that the respective lengths of the proximal section 20 and distal section 22 may vary. It can also be appreciated that while the proximal section 20 and distal section 22 have been described as having respective long axes 30 and 32, respectively, that the proximal section 20 and the distal section 22 can form a continuous bowed section where one or both of the proximal section 20 and distal section 22 form a curved profile.

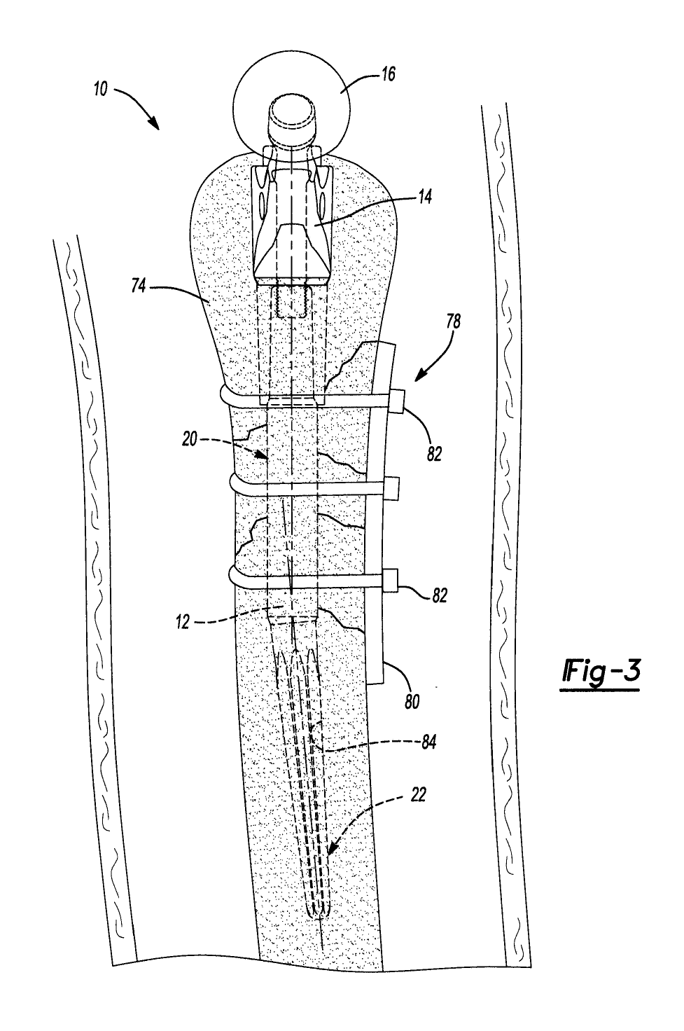

The proximal section 20 may be coated with porous material 38, such as, but not limited to, Regenerex® offered by Biomet, Inc., of Warsaw, Ind. Other porous materials can include plasma spray, grit blasting, etc. The porous material 38 covers substantially all of the proximal section 20 from the male taper end 24 to the start of the distal section 22. The distal section 22 can include a plurality of longitudinally extending splines or flutes 40 positioned radially about the distal section 22. The distal section 22 can be tapered from (or near) its transition from the proximal section 20 to a distal tip 42. The distal section 22 can also be slotted. A surface of the distal section 22 can be roughened such as by grit blasting. The stem 12 can be formed of a rigid biocompatible material, such as titanium for example. Other materials are contemplated. The stem 12 can be particularly suitable for providing favorable characteristics when implanted into an intramedullary (IM) canal 84 (FIG. 3) of a femur. In this regard, the stem 12 can utilize mechanical fixation with the tapered splines 40 to inhibit rotation and subsidence of the stem 12. Furthermore, the proximal section 20 of the stem 12 can incorporate porous coating 38 to encourage bony ingrowth for biologic fixation of the non-intact portion of the femur to heal into.

The proximal femoral body 14 can generally include a neck 44 that has a proximal end 46 including a male taper 48. The proximal femoral body 14 can further include a distal end 50 that has a bore 52. The bore 52 can include a female taper 54. The male taper 48 of the proximal end 46 can be configured to mate with a female taper 60 provided in the femoral head 16. In one example, the male taper 48 can mate with the female taper 60 as a Morse taper connection. It will be appreciated that in other examples, the male taper may be formed on the proximal femoral body 14 and the female taper formed on the stem 12. A threaded portion 64 can be formed in an opening 66 on the proximal femoral body 14.

Assembling the stem 12 with the proximal femoral body 14 according to one example will now be described. The male taper end 24 of the stem 12 can be inserted into the female taper 54 of the proximal femoral body 14. Further advancement of the male taper end 24 into the female taper 54 can encourage a Morse taper connection or taper-fit connection. A fastener 70 can optionally be advanced into threaded engagement with the blind threaded bore 26 of the male taper end 24 on the stem 12 to further encourage the male taper end 24 to be drawn into the female taper 54.

With additional reference now to FIG. 3, the modular femoral hip joint prosthesis 10 will be described in an implanted position within the IM canal 84 of a femur 74. The femur can have one or many fractures, such that portions of the femur 74 are generally intact and other portions are disconnected from adjacent bone.

In the exemplary configuration, the modular femoral hip joint prosthesis 10 can cooperate with a cerclage fixation assembly 78 that includes a plate 80 and a plurality of cables or monofilament wires 82. In one example, the plurality of cables or monofilament wires 82 can be wrapped around portions of the intact and non-intact portions of the femur 74. As explained, the femur 74 can have a fracture or series of fractures that may result in the femur 74 as collectively including one or a plurality of larger bone fragments, as well as one or a plurality of smaller bone fragments. It will be appreciated that in some examples, the surgeon may prepare the IM canal 84 of the femur 74 for receipt of the modular femoral hip joint prosthesis 10. Additional details of one exemplary method of preparing the femur 74 may be found in commonly owned and copending U.S. patent application Ser. No. (Attorney Docket No. 5490-000797) entitled “Method and Apparatus for Implanting a Modular Femoral Hip” filed concurrently herewith, which is hereby expressly incorporated herein by reference.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A modular femoral hip joint prosthesis comprising:
a proximal femoral body having a distal end and a proximal end, the proximal end being adapted to mate with a femoral head; and
a femoral stem having a proximal section and a distal section, the femoral stem being adapted to matingly connect with the distal end of the proximal femoral body, the proximal section having a porous metal coating adapted to facilitate boney ingrowth, the distal section having at least one spline formed along an outer surface.

2. The modular femoral hip prosthesis of claim 1 wherein the proximal section of the femoral stem extends along a first axis and wherein the distal section of the femoral stem extends along a second axis, wherein the first and second axes are non-parallel.

3. The modular femoral hip prosthesis of claim 2 wherein the distal section of the femoral stem tapers toward a distal tip.

4. The modular femoral hip prosthesis of claim 3 wherein the first and second axes extend at an angle substantially between 2 and 10 degrees.

5. The modular femoral hip prosthesis of claim 4 wherein the angle is 5 degrees.

6. The modular femoral hip prosthesis of claim 4 wherein the proximal section of the femoral stem includes a male taper end.

7. The modular femoral hip prosthesis of claim 6 wherein the distal end of the proximal femoral body includes a female taper.

8. The modular femoral hip prosthesis of claim 7 wherein the male taper end of the femoral stem is adapted to be received in the female taper of the proximal femoral body.

9. The modular femoral hip prosthesis of claim 8, further comprising a fastener that is operable to extend into an opening in the proximal femoral body and threadably mate with a threaded bore formed on the male taper end of the femoral stem.

10. A modular femoral hip joint prosthesis comprising:
a one-piece femoral stem having a proximal section and a distal section, the proximal section adapted to matingly connect with a proximal femoral body and having a porous metal coating that covers substantially all of the proximal section and is adapted to facilitate boney ingrowth, the distal section having a plurality of longitudinally extending splines formed along an outer surface, wherein the distal section tapers toward a distal tip.

11. The modular femoral hip prosthesis of claim 10, further comprising a proximal femoral body having a distal end and a proximal end, the proximal end being adapted to mate with a femoral head and the distal end being adapted to mate with the proximal section of the femoral stem.

12. The modular femoral hip prosthesis of claim 10 wherein the proximal section of the femoral stem extends along a first axis and wherein the distal section of the femoral stem extends along a second axis, wherein the first and second axes are non-parallel.

13. The modular femoral hip prosthesis of claim 12 wherein the distal section of the femoral stem is at least partially roughened.

14. The modular femoral hip prosthesis of claim 13 wherein the first and second axes extend at an angle substantially between 2 and 10 degrees.

15. The modular femoral hip prosthesis of claim 14 wherein the angle is 5 degrees.

16. The modular femoral hip prosthesis of claim 14 wherein the proximal section of the femoral stem includes a male taper end.

17. The modular femoral hip prosthesis of claim 16, further comprising a proximal femoral body having a distal end that includes a female taper.

18. The modular femoral hip prosthesis of claim 17 wherein the male taper end of the femoral stem is adapted to be received in the female taper of the proximal femoral body.

19. The modular femoral hip prosthesis of claim 18, further comprising a fastener that is operable to extend into an opening in the proximal femoral body and threadably mate with a threaded bore formed on the male taper end of the femoral stem.
